(19) United States
(12) Patent Application Publication
(10) Pub. No.: US 2014/0295538 A1
(43) Pub. Date: Oct. 2, 2014

(54) DEVICE AND SYSTEM FOR MECHANICAL MEASUREMENT OF BIOMATERIAL

(71) Applicant: BROWN UNIVERSITY, Providence, RI (US)

(72) Inventors: Christian Franck, Providence, RI (US); Eyal Bar-Kochba, Providence, RI (US)

(21) Appl. No.: 14/303,663

(22) Filed: Jun. 13, 2014

Related U.S. Application Data
(63) Continuation of application No. PCT/US2012/069779, filed on Dec. 14, 2012.
(60) Provisional application No. 61/576,008, filed on Dec. 15, 2011.

(51) Int. Cl. G01N 33/50

(52) U.S. Cl. CPC G01N 33/5005
USPC 435/288.7

(57) ABSTRACT

A test device applies a defined mechanical load to a soft biomaterial such as a cell culture and a microscope forms a volume image data set showing the strain field or displacement field occurring in the medium. The data set is processed to determine fundamental mechanical properties of the cell, its interaction with the surrounding medium, or its responses loading or deformation of its surrounding medium. The device may also be used to calibrate or determine fundamental mechanical properties of the medium. The device includes a linear actuator that bears against the specimen, and adapted for a volume imaging device such as a scanning laser confocal microscope that forms a volume image data set of the specimen. The specimen may be supported in a Petri dish and is preferably imaged from below by an inverted microscope. Preferably the actuator device attaches to or forms the specimen stage of microscope. Digital correlation of volumes in the data set allow computation of modulus, stress distribution and other mechanical characteristics of cell-matrix interactions, as well as mechanical properties of the cell and the matrix in response to changing loads, evolving chemical or ionic environment and growth phases. The test device may be operated to measure local mechanical parameters, to evaluate or design tissue-engineered implants, and to explore the mechanical properties of tissues, cells and cellular processes a micrometer scale with high accuracy.

### RELATED APPLICATION

This international application claims the benefit of U.S. provisional application Ser. No. 61/576,008 filed Dec. 15, 2011 in the U.S. Patent and Trademark Office, entitled “Test device for mechanical properties of soft biomaterial”, which is incorporated by reference herein in its entirety.

### FIELD OF INVENTION

Equipment for measurement, investigation, screening, or observation of biomaterial.

### BACKGROUND

The present invention relates to equipment for investigation, screening or observation of biomaterial such as tissue, cells and biomatrix, and to methods of using the equipment.

Cell mechanics and adhesion are widely recognized to play a central role in cell-tissue interactions and cellular functioning but their exact nature and mechanism are far from understood. Cells have been shown to mechanically probe and feel their surrounding microenvironment by applying displacements and exerting physical forces on their extracellular matrix. These phenomena have traditionally been investigated within a biologically-driven qualitative or biochemical framework. Over the last few decades, however, a paradigm shift has emerged from qualitative descriptions to a quantitative understanding of cellular mechanics; this has come about by introducing physical models and engineering-based measurement techniques to cell biology. Since then, several studies have identified cellular responses to local mechanical gradients. Such observations suggest the importance of developing a quantitative understanding of the magnitude, direction and duration of forces acting on cells, and of cell-generated forces as they contribute to such biological processes as wound healing, muscle growth, tissue assembly, and embryonic development. However the measurement of forces and of mechanical characteristics on a cellular scale remains difficult and is often approached on an ad hoc basis that yields only non-calibrated comparative estimates as measurements.

Thus is a there need for an apparatus and a methodology for repeatably and accurately measuring, and for creating quantifiable conditions for observing, mechanical properties of cells, cellular interactions and cellular responses.

There is also a need for such apparatus and protocols for inducing, observing, displaying and quantifying cellular interactions, such as strain dependent interactions, and for studying dynamic changes in properties of a cell or tissue under controlled conditions of stress or strain, in different media and/or culture environments.

### SUMMARY OF INVENTION

The present invention provides an apparatus and methods for accurate localized measurement of the mechanical properties and/or processes or interactions of a biomaterial such as a cell, a cultured tissue or a supporting or surrounding matrix medium forming the growth matrix of a cell or tissue culture, and for measuring growth, activity or responses of cells in a deformable or mechanically loaded environment, such as an environment having an applied load, strain distribution or deformation field. In one aspect the apparatus includes an actuator attachable to or integrated with a digital volume imaging device, such that the device images the specimen and derives measurements local strain or mechanical properties of imaged cells, cellular processes or interactions with the surrounding medium while the actuator creates a defined strain field in the specimen. Measurements of specimen properties may also be taken under controlled or known hydration, growth or nutrient media or other applied conditions or parameters to determine their effect on the cell and its responses to the strain field. Mechanical properties of the medium and mechanical interactions of a cell are measured with high accuracy on a micrometer- or sub-micrometer scale.

An embodiment of the invention includes a press assembly that provides a piston force, to create a uniform deformation or strain field in a medium containing or supporting a soft biospecimen. The device may be calibrated by operating on control specimens, such as acrylamide gels of different degrees of cross-linking and having known modulus, having nanoparticle markers distributed in the medium. The markers are tracked in each volume element or voxel by the volume imaging device to map the strain field induced by a given load, and may be re-imaged at one or more times to observe and quantify a response (such as movement of a cell) in the strain field, or to measure mechanical effects induced by or associated with movement of a cell in the medium, such as the magnitude, direction or distribution, and range of tensile or other forces exerted by the cell. The device is adapted to also make or permit dynamic observations in the field of view, of the effect of a defined load or impulse applied by the piston upon cellular processes of the biospecimen in the imaged volume.

According to this aspect of the invention, the press or actuator assembly includes, mounts upon or is otherwise adapted for integration with the specimen-imaging stage of a tomographic or sectioning imaging device such as a scanning laser confocal microscope, which provides a digital image of a volume including the specimen, thus allowing observation of the specimen while it is subjected to a controlled load or impulse. The device may image a volume image field containing cells and matrix material, and the observations may include dynamic or time-resolved imaging of a cellular growth, movement or other response to the applied load, or of a cellular process or interaction between one or more cells and the surrounding medium. According to this aspect, a processing system operates on image data to compute and display localized stresses, strain or forces in the sample under observation induced by or associated with cell movement and interactions of a cell with the surrounding medium, or may be operated to accurately quantify mechanical characteristics and interactions of a cell. Digital volume correlation determines the homogeneous deformation caused by the applied load, and further computations computationally derive mechanical properties such as the modulus of, or tensile, compressive or traction forces exerted by the cells themselves under the applied load or stimulus and defined culture conditions. The image data may also be processed to yield mechanical properties of the specimen induced by changing one or more conditions such as hydration, ionic content or matrix composition or load, or to screen for cells or tissue cultures that achieve a desired strength, modulus or other mechanical characteristic.

Attached to and incorporated by reference in the inventors' priority application (U.S. provisional application Ser. No. 61/576,008 filed Dec. 15, 2011) are two documents Appendix A and Appendix B, describing a prototype mechanical testing device, and reference is made to the bibliography of that Appendix A for general descriptions of the processes for measurement of strain fields by tracking imaged nanoparticles. The inventors have confirmed calibration of the testing device on acrylamide gels of known modulus and its operability for the purposes set forth in the original proposal on soft biomaterials to effect accurate measurements of mechanical characteristics of biological structures, materials and interactions on a microscopic scale, and for introducing a uniform axial strain field in a sample under study, observing cellular responses and mapping local mechanical interactions of the a cell in the field.

### BRIEF DESCRIPTION OF THE FIGURES

These and other features of the invention will be understood from the description herein and appendices hereto, the claims appended hereto and the Figures of an embodiment, wherein

FIG. 1A and 1B show two versions of a prototype test device of the invention for applying axial compression to a soft biospecimen on a microscope stage;

FIG. 2A shows the device of FIG. 1B mounted on a microscope imaging stage adapted to hold a culture dish or gel sample for monitoring the volumetric strain field and forming an image dataset to derive mechanical parameters of an observed specimen and its interactions;

FIG. 2B shows a gel cell and cover slip for inverted microscope imaging on the stage of FIG. 2A;

FIG. 3 illustrates an inverted microscope configured with an incubation chamber for observing cell culture in a gel medium;

FIG. 3A illustrates the microscope of FIG. 3 having a linear actuator positioned to provide a horizontal force or impulse to the culture medium;

FIG. 4 illustrates 100 micron impact displacement profiles for impulses of different duration measured with the linear actuator of FIG. 1A;

FIG. 5 illustrates the uniform strain field measured with the invention under a static load using digital volume correlation of particle displacements in the image volume;

FIG. 5A shows a representative image of the particles in an image field; and

FIG. 6 shows the measured three-dimensional displacement field induced in a culture medium by cell locomotion on the surface of the medium.

### DETAILED DESCRIPTION

In accordance with one aspect of the invention, actuator device applies axial compression to a transparent medium or culture containing nanoparticles to derive a measurement of a mechanical parameter of interest. A system operates with a scanning laser confocal microscope (SLCM) that performs microscopic volume imaging and correlation of the nanoparticles in digital volume images (voxels) to map a uniform deformation of the medium. As applied to a biomaterial specimen, the system provides an accurate measure of the mechanical parameter of the matrix or cellular material appearing in the volume image and collectively gives an accurate tomographic map of the actual strain at depth. The defamation may be a result of an impulse applied by the actuator, a static compressive load or other deformation-causing actuation applied at the surface of the medium to introduce a uniform axial strain field over a region of the specimen. Furthermore, cell-matrix interactions, such as traction forces exerted by a moving cell surrounded by the medium, may be detected as localized variations in the particle displacement field. In a control medium the strain field resulting from cell locomotion, as evidenced by the imaged particle distribution or displacement vectors proximate to the cell in an image data set, may be determined without actuator loading to provide an accurate measure of the cellular mechanics of interest. Alternatively, the actuator may first be operated to measure the modulus and strain distribution of an non-calibrated culture medium, and the voxels in the vicinity of a cell then processed to detect and quantify the cell-matrix interaction.

The method is highly accurate. For example, when the SLCM provides a digital volume image with a voxel dimension of one- or two- micrometers, and the medium is loaded with nanoparticles to provide about ten to fifty particles per voxel, displacement fields of sub-voxel accuracy are defined by correlation of the positions of corresponding particles in two successive images. When the modulus of the medium itself has been sufficiently characterized, which may be done by digital volume correlation of images taken under known load or impulse, and by comparing particle displacements to those occurring in a control the medium, e.g., a polymer or gel having a defined modulus and level of cross-linking, then the digital volume correlation of particle displacements induced in the medium by events such as the traction forces of cell movement, localized stresses or forces may be quantitatively converted to a measure of the tensile forces exerted by, or the work performed by, the cell or cellular process during movement.

Apparatus of the invention newly enables in vitro measurement, dynamically and accurately, of mechanical properties and interactions of small structures and at high resolution, as well as biomaterial baseline, diagnostic and screening measurements. The apparatus and method may be applied to detect, monitor or screen for changes in modulus of a culture medium or biomaterial such as collagen in response to repetitive impulses applied by the actuator, or as a function of other stress, nutrient composition, aging, hydration or other condition. Other effects may now be directly measured and observed, such as the strength of intracellular binding, or the magnitude and direction of cell migration cell in various mechanically-characterized surroundings; or effects of mechanical loading, impulse or other interactions on cell movement adhesion forces in cell-cell, free, anchored or stratified culture conditions in a sample biomaterial or cultured tissues.

In an embodiment, image analysis, particle-tracking and computational software may be integrated with actuator controls and microscope scanning/display controls so that a measured parameter, strain distribution and/or other mechanical measurand or construct is displayed and overlaid on the sample image. Such imaging and measurement may be performed in time-progression to elucidate an interaction of a cell with the surrounding matrix during growth, mobility or other process. The preferred microscopy instrument for imaging a biomaterial sample contacted by the actuator device is a scanning laser confocal microscope (SLAC microscope) capable of forming a tomographic digital volume image data set, that is a collection of images of thin focal sheets or layers of the observed specimen. The SLAC microscope may be a two- or three-color microscope, that internally redirects different wavelengths to different detector/processor arrays, for example using one or more wavelength selective filters or beam splitters, so that, for example red fluorescent nanoparticles may be efficiently tracked and analyzed in the matrix volume by one sensor/analysis component, while a second component receives a wavelength used for cell imaging, uptake studies or other distinct wavelength. Depending on factors such as the size of the observed field and the desired imaging and processing speed, a suitable SLAC microscope may be implemented using integrated lighting/scanning/detecting micromechanical photonic chips or chip arrays, a multi-aperture Nipkow disk scanner, and/or other suitable arrangement of component laser, scanner, splitter, detector and positioning elements.

A basic description of processing techniques for deriving and displaying a displacement field or deformation from the acquired image data set may be found in the paper Franck, C., S. Hong, S. A. Maskarinec, D. A. Tirrell, and G. Ravichandran, Three-dimensional full-field measurements of large deformations in soft materials using confocal microscopy and digital volume correlation. Experimental Mechanics, 2007. 47(3): p. 427-438; that paper is incorporated herein by reference in its entirety. When applied to a set of standardized control media, such as polyacrylamide gels of known concentration and defined Young's modulus, the linear stress-strain relationship allows the direct calculation of forces acting in the region from the observed displacement field. From another perspective, when a culture medium of known modulus and carrying nanoparticle markers is provided, then if displacement of markers is observed under static conditions in digital volume imaging in the vicinity of a cell in the medium by scanning laser confocal microscopy, the displacements of markers reflect, and thus may be computationally converted to cell-generated stresses and forces generated by migration, locomotion and other cellular functions. The culture medium may be compounded to have a modulus and particle loading which optimize the range and magnitude of these cell-generated effects, providing image- or measurement- enhancement similar to the enhancements achieved in conventional light microscopy with contrast agents, refractive index media, interferometric effects. One derived mechanical measurement protocol that may be used on image dataset of biospecimens may be an extension to three dimensions of the processing described in the paper Franck C, Maskarinec S A, Tirrell D A, Ravichandran G (2011) Three-Dimensional Traction Force Microscopy: A New Tool For Quantifying Cell-Matrix Interactions. PLoS 6(3):e17833. doi:10.1371/journal.pone.017833, to which reference is made for technical details. To the extent alloable under local patent practice, that paper is also incorporated herein by reference in its entirety.

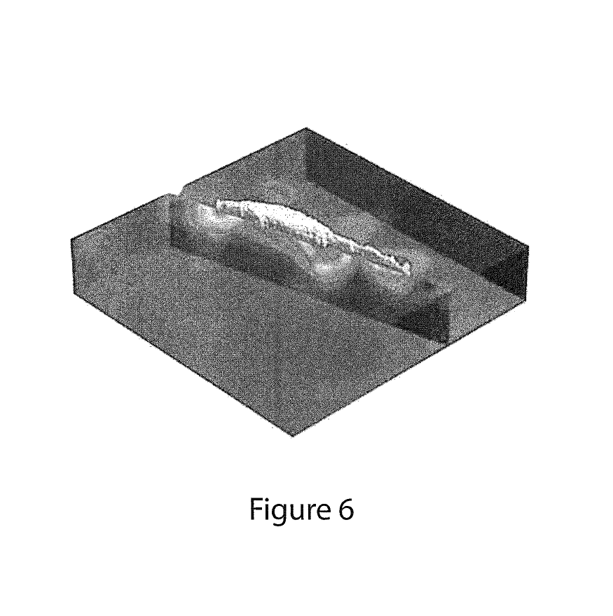

FIG. 6 shows a single fibroblast cell imaged by thsuch traction force microscopy process during cell movement, with a contour map showing the magnitude of the three dimensional displacement field induced in the support medium. Color contours are shown in the original revealing both pushing (compressive) and pulling(stretching) effects on the medium during cell locomotion along a surface. The aforesaid paper employed a calibrated culture medium of known modulus. Significantly, the actuator of the present invention is adapted for use on the stage of a scanning laser confocal microscope, and allows the creation of a uniform axial strain field in the culture or specimen, with observation of the induced deformation field of the nanoparticle-loaded culture or embedding medium, and accurate calculation of the modulus of the medium, so that accurate traction force measurements and other mechanical measurements can now be accurately effected in vitro on a broad range of biological specimens in arbitrary media, and permitting measurement and mapping of micro-scale mechanical forces and interactions of biological materials in three dimensions.

Thus the mechanical testing device of the invention is specifically adapted to receive a soft biosample and to provide a defined strain field in the soft biomaterial and to characterize and to quantitatively study mechanical parameters of cell-tissue interactions or strain-related processes or changes with micrometer or sub-micrometer resolution. The device thus operates as a controlled mechanical stimulation platform for biospecimen observation and biomechanical data recording.

Protocols for use of the platform may employ three-dimensional full-field imaging. They may apply a digital volume correlation as set forth in the above-cited papers, and may apply further transformations operating on an acquired image data set to compute the localized mechanical forces, modulus and other mechanical parameters associated with cell-applied deformations in the soft biomaterial or tissue culture specimen, thus providing accurate and quantifiable measurements of the mechanical descriptors of the tissue and its interactions.

An embodiment of the present invention may include a load frame with an actuator for applying a load to a specimen supported on a sample stage, and operates with an imaging device directed at the sample stage for imaging the sample under the defined strain distribution. In a proof-of principle prototype the actuator is a linear actuator which drives a piston to apply a force to a ram or compression plate at the top surface of a specimen under observation. For example, the specimen may be a cylindrical plug of culture material cast in a mold and carried in a Petri dish, and the compression plate may be a microscope cover slip, for example No. 0 or other cover slip. The cover slip may be contacted by the actuator in a central region of the slip, and spreads the force exerted by the piston over an area—for example a one centimeter disc area—at the top surface of the biosample. The thin cover slip may bend and slightly deform at the region of piston contact, under the contemplated loading, thus inducing a more uniform strain field in the underlying biospecimen that would arise from direct contact of the piston face alone. The cover slip may be pretreated, for example glutaraldehyde treated and/or precoated with a thin layer of the matrix medium, so as to bear against or even attach to the underlying specimen in a non-slip fashion. In an embodiment, the actuator piston may have a rounded tip—for example of two millimeters diameter.

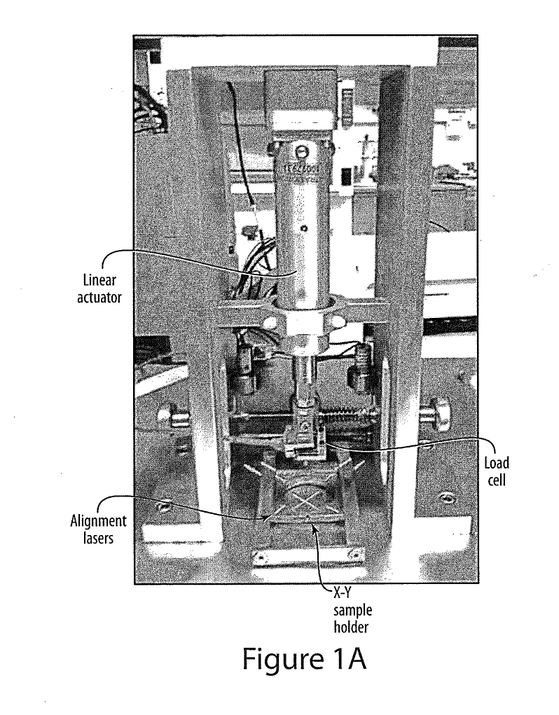

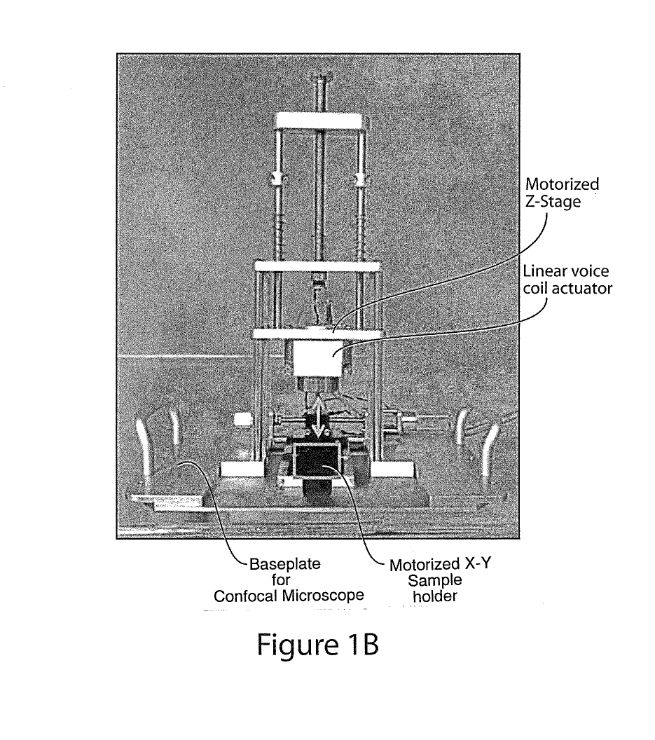

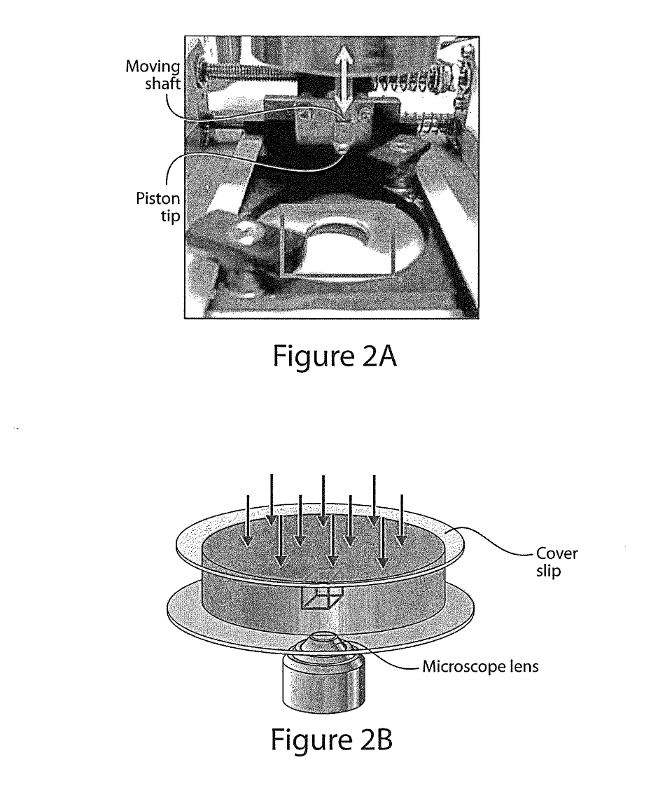

The linear actuator may be a linear motor, or may be a voice coil; in either case, a suitable actuation signal control circuit is provided to set the magnitude, stroke and duration of load or impulse delivered by the actuator. FIGS. 1A and 1B show mechanical prototypes of a linear motor driven actuator, and a voice coil driven actuator.

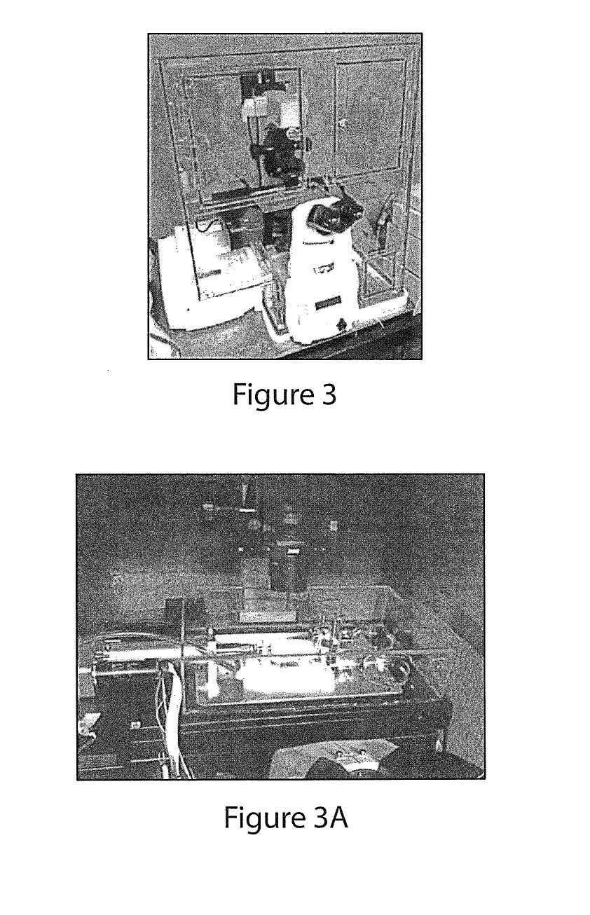

When used with a scanning laser confocal microscope to form the digital volume image data set, the microscope is preferably an inverted microscope, as shown in FIG. 3, and the piston assembly is mounted above the stage to push downward. Alternatively the actuator may be mounted next to the stage as shown in FIG. 3A to push horizontally sideways on a specimen, such as a gelatin culture medium, while the microscope scans the volume from below. Such side-actuation may be used, for example, to model strain-induced migration of cells in a stratified tissue while the microscope provide a high resolution cell imaging as well as tomographic data set from which local strain distribution is derived by digital volume correlation techniques. The microscope stage may be fitted with a cell incubation chamber, as shown in FIG. 3 for performing observations and measurement on cells under dynamic conditions of growth or movement. Systems of the present invention may be applied to cultures, wherein culture images are used to evaluate or screen media, nutrients or cell lines for health-related aspects of cell mobility, contractile strength or other characteristics; or to identify concentration-, viscosity-dependent or other factors useful in optimizing cultures in industrial production processes.

Preferably the imaging device is a high-resolution device, capable of micron or sub-micron resolution, for example 50 nm, 100 nm or 200 nm resolution. Higher resolution allows the strain distribution to be more accurately determined using the digital volume correlation as described in the papers referenced above, e.g., wherein inhomogeneities in the form of small fluorescent styrene beads or nanoparticles may be added to the medium in a sufficient concentration to allow an efficient and effective the correlation process and derivation of strain field or other measurement from the image data set with the level of resolution or accuracy required for a contemplated observation or achievable with the available SLAC microscopy system. For cell mobility studies, the long times involved (e.g., minutes or hours of slow movement) allow for effective correlation and computation of many small-voxel data sets between successive images.

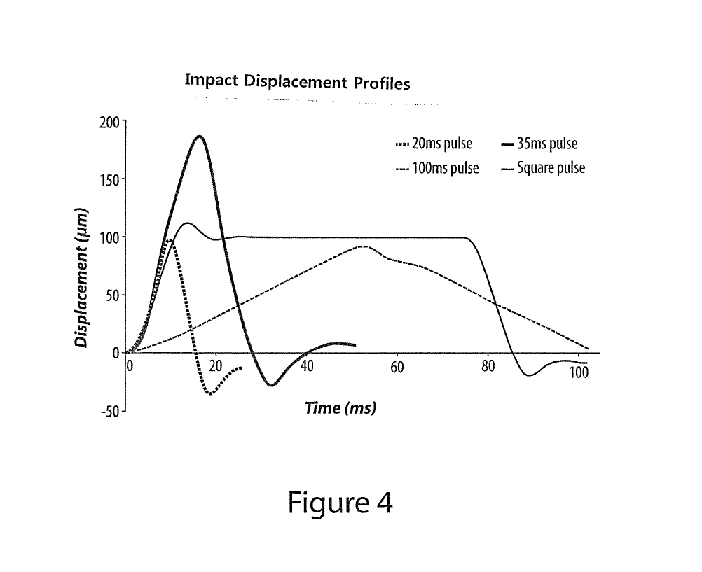

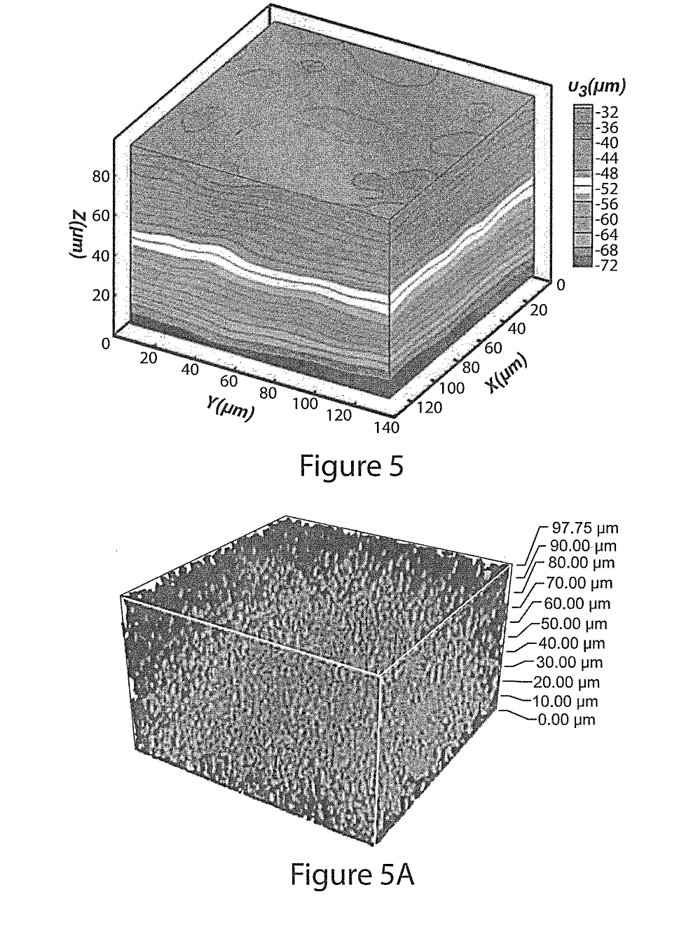

Preferably a load cell (FIG. 1A) is also provided, positioned to measure the force applied by the linear actuator, so that each experiment can be readily set up and, if necessary at all, quickly calibrated. A user interface such as a computer or control chip with suitable software allows the user to set, to monitor and to record the actuator position and the force applied by the actuator. Preferably the computer continuously monitors and records actuator position and load as the specimen is imaged and recorded so that successive images are readily registered, and the digital volume image data set, images of cells, and applied load or impulse data may be analyzed later and need not be processed in real time. FIG. 4 illustrates 100 micron impact displacement profiles for the linear actuator of FIG. 1A with 20-, 35- and 40- millisecond impulse and 100 millisecond step actuation. FIG. 5 illustrates the uniform strain field over a depth, as measured with the invention under a static load by digital volume correlation of the displacement of imaged marker particles (FIG. 5A) in the image volume.

The results from the mechanical testing device can be used in the development and prediction of cell motility and cell adhesion studies; such results are expected to advance our understanding of processes and effects in the emerging fields of tissue engineering and regenerative medicine which were previously incapable of observation or of accurate measurement. Results confirm that the test device has successfully characterized tissue-mimicking polyacrylamide hydrogels ranging in their Young's modulus from 500-50,000 Pa, thus representing a large portion of the typical stiffness range found in the human body and ideal for cell-tissue interaction studies. A user interface for setting the test conditions and processing the image data set to display derived mechanical stress or other fields associated with the cell-matrix interactions is sufficiently intuitive to permit graduate and undergraduate students from various backgrounds to operate the machine to extract accurate mechanical data associated with the image field and cellular events. For example the magnitude of local strain may be coded in different colors, so that a uniform axial deformation is represented as a stack of parallel sheets of different color and thickness (FIG. 5), and the strain induced in the surrounding media by a cell's movement appears in different frames as corresponding regions of color overlaid on the image of the cell. The observed displacements, and derived strain and stress measurements have been found to be quite accurate and repeatable thus allowing more advanced study of nutritional, genetic and other factors affecting cellular mechanics and cellular responses to mechanical challenges.

The invention and its application to a representative control specimens and biological samples being thus described, further features, advantages, breakthrough measurement opportunities and protocols for using the described apparatus will occur to those skilled in the art. Thus, for example, the device may be employed to apply repetitive loading or mechanical stress to cell cultures of different cell lines to identify an optimum line for replacement or transplantation; the device may be applied to quantify the strength or suitability of, or to perform quality-assurance testing of a cultured or bioengineered bioimplant; may be operated to apply impulses of varying magnitude and observe the related cellular responses or damage to elucidate the cellular mechanisms of traumatic injury or repair, and such observations may be incorporated in various sensor or alarm devices for use in a hospital, battlefield or clinical setting. The linear actuators of FIGS. 1A and 1B may be positioned to induce rotational, rather than axial impulses and the effects of mechanical shear in media of calibrated modulus may be then be accurately determined and responses evaluated in layered tissues; the actuator and digital volume dataset analysis for measurement of mechanical parameters may be operated to achieve other mechanical measurements or observe mechanically-related aspects of cell growth, development, pathology or differentiation that have heretofore resisted measure or characterization.

What is claimed is:

1. System and apparatus for observational analysis of a biomaterial sample such as a cell, a tissue culture or a culture medium, the apparatus comprising
a load assembly including a specimen stage adapted for holding a biomaterial sample and an actuator for controllably applying a load to the sample so as to generate forces and strain in the sample effective to create a homogeneous deformation
the load assembly being mountable upon an imaging device, such that the imaging device forms an image of a volume image field including the sample
a processor operative on the image to computationally define said homogeneous deformation and determining an image data set of local mechanical properties, displacements and/or volume change under the applied load conditions in the volume image
wherein the apparatus is calibrated for operation such that the actuator creates said homogeneous deformation field with defined values and the imaging device images a correlated response or interaction of the sample.

2. Apparatus according to claim 1, wherein the apparatus includes a user-controlled means for setting one or more of the applied load and/or the modulus of a medium containing the sample such that observation of the sample indicates one or more of: a biological response of the sample to the deformation field, a measurement of a mechanical response of the sample to the deformation field, and a measurement of a mechanical property of a growth or motility action of the sample.

3. Apparatus according to claim 1, wherein the apparatus is operable to display or record in-situ micro-scale mechanical measurements of tissue, of a cell or of a cellular process.

4. Apparatus according to claim 1, wherein the imaging device is a confocal microscope or an inverted scanning laser microscope.

5. Apparatus according to claim 1, wherein the sample contains markers, and the device performs a digital volume element correlation of markers to derive a volumetric strain distribution in the sample.

6. Apparatus according to claim 1, wherein the apparatus numerical values or as a color field distribution over the volume image for correlation with an observed image of the sample.

7. Apparatus according claim 1, wherein the imaging data set is processed to produce a time sequence image of a living cell or cell culture while the actuator is controlled to subject the sample to defined local mechanical strain, stress, modulus or other mechanical condition.

8. Apparatus according to claim 1, wherein the image data set is processed to image and produce a time sequence measurement of a local cell-matrix interaction such as traction force.

9. Apparatus according to claim 1, wherein the imaging data set is processed to produce an image having micrometer or sub-micrometer resolution.

10. Apparatus according to claim 1, wherein the specimen stage supports the sample in a culture medium of defined modulus over an opening through which the microscope images a volume in the sample while the sample is subjected to the deformation.

11. Apparatus according to claim 1, wherein the apparatus is used to measure mechanical strain field or other property of a gel sheet of known modulus as a control for calibrating a measurement or response of the biomaterial sample.

12. A test device for performing measurements or observations of mechanical properties on a soft biomaterial, wherein the device adapts to a sample stage for supporting a soft biomaterial and includes an actuator for establishing a defined mechanical load on the biomaterial, such that volume imaging of the biomaterial on the sample stage measures a mechanical property, response or interaction of the biomaterial to the defined mechanical load.

13. A test device according to claim 12, wherein the measure of a mechanical property includes a time-resolved measure of a change or a response to a static or dynamic pressure exerted by the actuator.

14. A test device according to claim 12, wherein the test device is mountable on an imaging device for imaging the biomaterial while the biomaterial is subject to the defined mechanical load such that the image forms an image dataset for computational derivation of a desired mechanical measurement such as a modulus, traction force or other mechanical property or cell-matrix interaction of the biomaterial in a strain field introduced by the actuator.

15. A test device according to claim 12, wherein the biomaterial is transferable from the test device to an imaging device after being cultured under defined load conditions or strain field, to measure or observe biological properties of the biomaterial resulting from having been cultured in the defined load conditions or strain field.

16. A test device according to claim 12, wherein the test device is operated to subject the biomaterial to a load or impulse and the biomaterial is imaged to determine a response, such as cell death, dysfunctional tissue growth or other aberrant response constituting a critical damage response to mechanical trauma conditions.

17. A test device according to claim 12, wherein the test device is operated to subject the biomaterial to a load or impulse and the biomaterial is imaged or otherwise tested to observe or otherwise determine a response, such as enhanced tissue modulus or strength, or tissue survivability, constituting a desirable response so as to screen the biomaterial when cultured under said load or impulse condition for suitability for implantation, surgical or reconstructive use.

18. A test device according to claim 12, wherein the test device is operated to calibrate a nanoparticle-containing support or culture medium, and the biomaterial is imaged in said medium by a scanning laser confocal microscope as it moves to measure mechanical forces exerted by the biomaterial on the medium.
